(19) United States
(12) Patent Application Publication
(10) Pub. No.: US 2006/0188555 A1
(43) Pub. Date: Aug. 24, 2006

(54) Therapeutic peptide formulations with improved stability

(76) Inventors: Micheal Cormier, Mountain View, CA (US); Mahmoud Ameri, Fremont, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/336,134

(22) Filed: Jan. 19, 2006

Related U.S. Application Data
(60) Provisional application No. 60/645,996, filed on Jan. 21, 2005.

(51) Int. Cl. A61K 38/28; A61K 38/21; A61K 38/22; A61K 38/19; A61K 38/18; A61F 13/02; A61L 15/16

(52) U.S. Cl. 424/448; 514/3; 424/85.1; 424/85.6; 514/12; 604/500

(57) ABSTRACT

Compositions of and methods for formulating and delivering peptide, polypeptide and protein therapeutic agent formulations having enhanced physical stability, and wherein fibril formation is minimized and/or controlled, to yield a consistent and predictable composition viscosity. The compositions of and methods for formulating and delivering peptide, polypeptide and protein therapeutic agents of the present invention further facilitate their incorporation into a biocompatible coating which can be employed to coat a stratum-corneum piercing microprojection, or a plurality of stratum-corneum piercing microprojections of a delivery device, for delivery of the biocompatible coating through the skin of a subject, thus providing an effective means of delivering the peptide therapeutic agents.

### CROSS REFERENCE TO RELATED APPLICATIONS

This application claims the benefit of U.S. Provisional Application Ser. No. 60/600,638, filed Aug. 10, 2004.

### FIELD OF THE PRESENT INVENTION

The present invention relates generally to peptide, polypeptide and protein therapeutic agent compositions and methods for formulating and delivering such compositions. More particularly, the present invention relates to compositions of and methods for formulating and delivering physically stabilized peptide, polypeptide and protein therapeutic agent compositions by controlling the tendency of such therapeutic agent compositions to form fibrils in solution.

### BACKGROUND OF THE INVENTION

A great number and variety of peptide, polypeptide, and protein therapeutic agents are known in the art to have therapeutic benefits when delivered appropriately to a patient having a condition upon which such therapeutic agents can exert a beneficial effect. These therapeutic agents comprise several broad classes, including, but not limited to: hormones, proteins, antigens, repressors/activators, enzymes, and immunoglulins, among others. Therapeutic applications include treatment of cancer, hypercalcemia, Paget's disease, osteoporosis, diabetes, cardiac conditions, including congestive heart failure, sleep disorders, Chronic Obstructive Pulmonary Disease (COPD) and anabolic conditions, to name a few.

In the art, formulating such peptide, polypeptide, and protein therapeutic agent formulations in a therapeutically effective and commercially viable manner has been problematic, due in part, to the tendency of many peptide, polypeptide and protein therapeutic agents to form fibrils when present in a therapeutic-effective concentration. Fibril formation in formulations of peptides, polypeptides or proteins has been regarded as somewhat unpredictable. Fibril formation may occur soon (within hours) after formulation and may thus adversely impact manufacturability of a therapeutic agent formulation containing the peptide, polypeptide and protein. Fibril formation can also occur in the final product after manufacture, leading to a decrease in shelf life.

The formation of fibrils in formulations of peptide, polypeptide and protein affects the physical stability of the formulation by causing the formulation viscosity to change (e.g., increase) over time. The kinetics of such change can be difficult to predict and, hence, account for in the process of commercially manufacturing the peptide, polypeptide or protein therapeutic agent formulations.

References have been published which discuss the causes and effects of protein fibrillation in vivo. For example, Lomakin et al, Proceedings of the National Academy of Sciences, Vol 93, pp 1125-1129 (February 1996) addresses fibril formation in human actin tissue.

Neither the above noted publication, nor any other known reference, however, disclose a formulation of, or technique for, physically stabilizing formulations of peptide, polypeptide, or protein therapeutic agents, in particular, mitigating or eliminating fibril formation, and resultant unwanted changes in formulation viscosity. In particular, neither the noted publication, nor any other known reference disclose a formulation of, or technique for, physically stabilizing peptide, polypeptide and protein therapeutic agents by formulating the therapeutic agents with an appropriate counterion, or a mixture of counterions, which impart to the formulation stability against undesired fibril formation, and consequent increase or change over time of formulation viscosity.

Improved physical stability of such therapeutic formulations of peptide, polypeptide and protein therapeutic agents provides not only the benefit of an increased storage or shelf life for the therapeutic agent itself, but enhances efficacy in that once stabilized in accordance with the compositions of and methods for formulating and delivering of the present invention, the therapeutic agents become useful in a greater range of possible formulations, and with a greater variety of therapeutic agent delivery means.

Therapeutic agents, such as peptides, polypeptides and proteins are typically administered orally, by infusion, by injection, and more recently, by transdermal delivery. The word “transdermal”, as used herein, is generic term that refers to delivery of an active agent (e.g., a therapeutic agent, such as a drug, pharmaceutical, peptide, polypeptide or protein) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Numerous transdermal agent delivery systems and apparatus have been developed that employ tiny skin piercing elements to enhance transdermal agent delivery. Examples of such systems and apparatus are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin, and thus enhance the agent flux. The piercing elements generally extend perpendicularly from a thin, flat member, such as a pad or sheet. The piercing elements are typically extremely small, some having a microprojection length of only about 25-400 microns and a microprojection thickness of only about 5-50 microns.

Recent improvements in transdermal agent delivery systems include systems, methods and formulations wherein the active agent to be delivered is coated on the microprojections instead of contained in a physical reservoir. This eliminates the necessity of a separate physical reservoir and developing an agent formulation or composition specifically for the reservoir. U.S. Patent Application Publication Nos. 2004/0062813 (Cormier et al), and 2004/0096455 (Maa et. al.), the disclosures of which are fully incorporated by reference herein, disclose compositions of and methods for formulating and delivering the active agent by coating the agent onto the microprojections.

The above U.S. patent applications note that the coating process should be carefully controlled and monitored to ensure that an effective amount of therapeutic agent is delivered. Factors important to achieving the therapeutic-effective dose include precisely controlling the thickness of the coating applied onto the surface of microprojections of the delivery device. As is known in the art, the desired thickness of the coating on the microprojections is dependent upon several factors, including the viscosity and concentration of the coating composition.

Accordingly, physical stabilization, especially maintaining the viscosity stability of the peptide, polypeptide, and protein therapeutic agent formulations over time, is an important step in assuring efficacy of the therapeutic agents, particularly when the mode of delivery of the therapeutic agent is via a transdermal delivery device having a plurality of microprojections coated with an agent containing biocompatible coating.

It would therefore be desirable to provide compositions of and methods for formulating and delivering peptide, polypeptide and protein therapeutic agents having enhanced physical stability.

It would be further desirable to provide compositions of and methods for formulating and delivering peptide, polypeptide and protein therapeutic agents wherein fibril formation is minimized and/or controlled.

It would be further desirable to provide compositions of and methods for formulating and delivering peptide, polypeptide and protein therapeutic agents wherein the minimization and/or control of fibril formation results in a consistent and predictable composition viscosity.

It would be further desirable to provide compositions of and methods for formulating and delivering peptide, polypeptide and protein therapeutic agents that exhibit maximal or optimal shelf lives.

It would be further desirable to provide peptide, polypeptide and protein therapeutic agents having enhanced physical stability, wherein the peptide, polypeptide and protein therapeutic agents are coated on a transdermal delivery device having a plurality of skin-piercing microprojections that are adapted to deliver the agent through the skin of a subject.

In accordance with the compositions of and methods for formulating and delivering physically and viscosity stable peptide, polypeptide and protein therapeutic agent formulations of the present invention, it has been found that the addition of an appropriate mixture of counterions to a therapeutic agent formulation substantially reduces or eliminates undesirable fibril formation, and consequent undesirable variations in formulation viscosity.

In accordance with the present invention, it is believed that fibril formation is a function of the secondary structure of the peptide, polypeptide or protein. Fibrils have been observed to grow as a function of time by what is believed to be an elongation, or a self-association, process. The alpha-helix configuration of certain polypeptides, for example, the growth hormone releasing factor (GRF) analog, TH9507, has been found by the inventors herein to lead to self-association, a crystallization-like process. Crystal formation occurs with compounds that can self-associate in repetitive patterns, which is possible only if the basic units, or molecules, are identical to each other.

The introduction of a mixture of two or more counterions to a peptide, polypeptide or protein solution makes the solution behave like a mixture of different peptides, which renders self-assembly very difficult, mitigating or eliminating fibril formation, and consequent undesirable increases in formulation viscosity.

It is therefore an object of the present invention to provide compositions of and methods for formulating and delivering peptide, polypeptide and protein therapeutic agents possessing enhanced physical stability.

It is another object of the present invention to provide compositions of and methods for formulating and delivering peptide, polypeptide and protein therapeutic agents wherein fibril formation is minimized and/or controlled.

It is another object of the present invention to provide compositions of and methods for formulating and delivering peptide, polypeptide and protein therapeutic agents wherein the minimization and/or control of fibril formation results in a consistent and predictable composition viscosity.

It is yet another object of the present invention to provide compositions of and methods for formulating and delivering peptide, polypeptide and protein therapeutic agents that have maximal or optimal shelf lives.

It is a further object of the present invention to provide peptide, polypeptide and protein therapeutic agents having enhanced physical stability, wherein the peptide, polypeptide and protein therapeutic agents are contained in a biocompatible coating that is disposed on a transdermal delivery device having a plurality of skin-piercing microprojections that are adapted to deliver the agent through the skin of a subject.

It is another object of the present invention to provide compositions of and methods for formulating and delivering peptide, polypeptide and protein therapeutic agent formulations wherein the formulations are stabilized with a counterion mixture.

It is further object of the present invention to provide methods for using a mixture of counterions to stabilize peptide, polypeptide and protein therapeutic agent formulations.

It is a further object of the present invention to provide methods for predicting and determining the effects of a mixture of counterions, upon the stability of peptide, polypeptide and protein therapeutic agent formulations, and wherein the methods permit a desired formulation viscosity to be accurately targeted, for example, in the manufacture of the therapeutic agent formulations.

### SUMMARY OF THE INVENTION

In accordance with the above objects and those that will be mentioned and will become apparent below, in one embodiment of the invention, there are provided compositions of and methods for formulating and delivering peptide, polypeptide and protein therapeutic agents that exhibit improved or optimal physical stability, and which improved or optimal physical stability enhances shelf life of formulations containing the therapeutic agents. The present invention also provides for compositions of and methods for formulating and delivering peptide, polypeptide and protein therapeutic agent formulations that exhibit improved or optimal physical stability, and which can accordingly be incorporated in a biocompatible coating that is coated onto a plurality of stratum corneum-piercing microprojections of a transdermal delivery device.

The present invention further provides predictive methods for assessing and/or determining the tendency of a given peptide, polypeptide or protein solution to form fibrils, and to provide appropriate mixtures of two or more counterions to inhibit, prevent or counteract the fibril formation, and wherein the methods permit the viscosity of the therapeutic agent formulations to be accurately targeted.

The present invention additionally provides predictive methods for evaluating, predicting and inhibiting peptide self assembly, based upon charge distribution, stoichometric and thermodynamic considerations.

In one embodiment of the present invention, the compositions of and methods for formulating and delivering peptide, polypeptide and protein therapeutic agent formulations are suitable for use with a variety of delivery means (e.g., systemic or local delivery), including oral (bolus), oral (timed or pattern release), infusion, injection, subcutaneous implant, pulmonary, mucosal (oral mucosa, ocular, nasal, rectal, vaginal), passive, active and balistic transdermal delivery. Other local delivery, such as treatment of otitis, skin, scalp, nail fungal, bacterial and viral infections, are also within the scope of the invention.

In a preferred embodiment, the compositions of and methods for formulating and delivering peptide, polypeptide and protein therapeutic agents are particularly suitable for transdermal delivery using a microprojection delivery device, wherein the peptide or polypeptide therapeutic agents are included in a biocompatible coating that is coated on at least one stratum-corneum piercing microprojection, preferably a plurality of stratum-corneum piercing microprojections of a microprojection delivery device.

In one embodiment of the present invention, the compositions of therapeutic peptides, polypeptides and proteins includes at least one of the following agents that can form fibrils under usual or particular conditions: ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH and VIP.

Further specific examples of therapeutic agents include, without limitation, growth hormone release hormone (GHRH), octreotide, pituitary hormones (e.g., hGH), ANF, growth factors, such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, and polypeptides and polypeptide analogs and derivatives derived from the foregoing.

In a preferred embodiment, the therapeutic peptide agent comprises a hormone. A particularly preferred hormone is Growth Hormone Releasing Factor (GRF) and analogs thereof, especially TH 9507. TH 9507 has proven valuable to treat sleep disorders, anabolic indications including muscle wasting in Chronic Obstructive Pulmonary Disease (COPD), and following certain surgeries. In this embodiment, it is particularly preferred to stabilize the GRF with a mixture of acetate and chloride counterions. The mole ratio of acetate to chloride is preferably in the range of about 0.2:1-5:1, more preferably, in the range of about 0.5:1-2:1. The mole ratio of the counterion mixture to peptide is preferably in the range of about 2:1-30:1, more preferably, in the range of about 4:1-15:1.

In accordance with one embodiment, the present invention comprises a peptide or polypeptide formulation wherein at least two counterions are associated with the peptide or polypeptide. The counterions of the therapeutic peptides or polypeptides are those that form pharmaceutically acceptable salts thereof. Thus, for peptides or polypeptides possessing a net negative charge, the counterion mixture should possess a net positive charge at the solution pH. For peptides or polypeptides possessing a net positive charge, counterion mixture should possess a net negative at the solution pH.

Where two counterions are employed, a mole ratio of the two counterions is preferably in the range of about 0.2:1-5:1, more preferably, in the range of about 0.5:1-2:1. Where three or more counterions are employed, the mole ratio of any individual counterion to the molar sum of the others is preferably in the range of about 0.1:1-2.5:1, more preferably, in the range of about 0.25:1-1:1. The mole ratio of the counterion mixture to peptide is preferably in the range of about 2:1-30:1, more preferably, in the range of about 4:1-15:1.

Examples of counterions suitable for formulation with net positively charged peptides or polypeptides include, but are not limited to, acetate, propionate, butyrate, pentanoate, hexanoate, heptanoate, levulinate, chloride, bromide, citrate, succinate, maleate, glycolate gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate and sulfonate.

Examples of counterions suitable for formulation with net negatively charged peptides or polypeptides include, but are not limited to, sodium, potassium, calcium, magnesium, ammonium, monoethanolamine, diethanolamine, triethanolamine, tromethamine, lysine, histidine, arginine, morpholine, methylglucamine, and glucosamine.

In another preferred embodiment, the resultant formulation of stable peptide, polypeptide and protein therapeutic agents, including the counterion mixture, is incorporated in a biocompatible coating used to coat at least one stratum-corneum piercing microprojection, preferably a plurality of stratum-corneum piercing microprojections, or an array thereof, or a delivery device. Typically, the coating process is carried out in a series of coating steps, with a drying step between each coating step, as disclosed, for example in U.S. Pat. Pub. No. 2002/0132054, to Trautman et al.; the disclosure of which is incorporated by reference herein.

In accordance with a further embodiment of the invention, an apparatus or device for transdermally delivering the stable peptide, polypeptide and protein therapeutic agents comprises a microprojection member that includes a plurality of microprojections that are adapted to pierce through the stratum corneum into the underlying epidermis layer, or epidermis and dermis layers, the microprojection member having a biocompatible coating disposed thereon that includes a formulation containing the stable peptide, polypeptide and protein therapeutic agents. In a preferred embodiment, the therapeutic agent comprises a Growth Releasing Factor (GRF) and analogs thereof. More preferably, in such embodiments, the therapeutic agent comprises TH 9507.

In accordance with one embodiment of the invention, a method for delivering peptide therapeutic agent formulations comprises the following steps: (i) providing a microprojection member having a plurality of microprojections, (ii) providing a stabilized formulation of peptide therapeutic agent; (iii) forming a biocompatible coating formulation that includes the formulation of stabilized peptide therapeutic agent, (iv) coating the microprojection member with the biocompatible coating formulation to form a biocompatible coating; (v) stabilizing the biocompatible coating by drying; and (vi) applying the coated microprojection member to the skin of a subject.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection array upon which a biocompatible coating having a peptide therapeutic agent formulation can be deposited;

FIG. 2 is a perspective view of the microprojection array shown in FIG. 1 with a biocompatible coating deposited onto the microprojections;

FIG. 2A is a cross-sectional view of a single microprojection taken along line 2A-2A in FIG. 1;

FIG. 3 is a schematic illustration of a skin proximal side of a microprojection array, illustrating the division of the microprojection array into various portions, according to the invention;

FIG. 4 is a side plane view of a skin proximal side of a microprojection array, illustrating the division of the microprojection array into various portions, according to the invention;

FIG. 5 is a side sectional view of a microprojection array illustrating an alternative embodiment of the invention, wherein different biocompatible coatings may be applied to different microprojections;

FIGS. 6A and 6B are phase-contrast photomicrographs of a polypeptide formulation of the prior art, showing fibril formation; and

FIGS. 7A and 7B are phase-contrast photomicrographs of a polypeptide formulation of the present invention, showing the absence of fibril formation.

### MODES FOR CARRYING OUT THE INVENTION

Before describing the present invention in detail, it is to be understood that this invention is not limited to particularly exemplified materials, formulations, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a therapeutic agent” includes two or more such agents; reference to “a microprojection” includes two or more such microprojections and the like.

### Definitions

The terms “peptide”, “polypeptide” and “protein” are used interchangeably herein. Unless otherwise clear from the context, the noted terms refer to a polymer having at least two amino acids linked through peptide bonds. The terms thus include oligopeptides, protein fragments, analogs, derivatives, glycosylated derivatives, pegylated derivatives, fusion proteins and the like.

The term “transdermal”, as used herein, means the delivery of an agent into and/or through the skin for local or systemic therapy.

The term “transdermal flux”, as used herein, means the rate of transdermal delivery.

The term “stable”, as used herein to refer to an agent formulation, means the agent formulation is not subject to undue chemical or physical change, including decomposition, breakdown, or inactivation. “Stable” as used herein to refer to a coating also means mechanically stable, i.e. not subject to undue displacement or loss from the surface upon which the coating is deposited.

The terms “therapeutic agent” and “agent”, as used herein, mean and include a pharmaceutically active agent and/or a composition of matter or mixture containing an active agent, which is pharmaceutically effective when administered in a therapeutic-effective amount. A specific example of a peptide therapeutic active agent is a GRF. It is to be understood that more than one “agent” can be incorporated into the therapeutic agent formulation(s) of the present invention, and that the terms “agent” and “therapeutic agent” do not exclude the use of two or more such agents.

The terms “therapeutic-effective” or “therapeutically-effective amount”, as used herein, refer to the amount of the therapeutic peptide agent needed to stimulate or initiate the desired beneficial result. The amount of the therapeutic peptide agent employed in the coatings of the invention will be that amount necessary to deliver an amount of the therapeutic peptide agent needed to achieve the desired result. In practice, this will vary widely depending upon the particular therapeutic peptide agent being delivered, the site of delivery, and the dissolution and release kinetics for delivery of the therapeutic peptide agent into skin tissues.

The term “coating formulation”, as used herein, means and includes a freely flowing composition or mixture, which is employed to coat a delivery surface, including one or more microprojections and/or arrays thereof.

The term “biocompatible coating”, as used herein, means and includes a coating formed from a “coating formulation” that has sufficient adhesion characteristics and no (or minimal) adverse interactions with the peptide therapeutic agent.

The term “microprojections”, as used herein, refers to piercing elements that are adapted to pierce or cut into and/or through the stratum corneum into the underlying epidermis layer, or epidermis and dermis layers, of the skin of a living animal, particularly a mammal and, more particularly, a human.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration. The microprojection member can also be formed in other known manners, such as by forming one or more strips having microprojections along an edge of each of the strip(s), as disclosed in U.S. Pat. No. 6,050,988, which is hereby incorporated by reference in its entirety.

Microprojection members that can be employed with the present invention include, but are not limited to, the members disclosed in U.S. Pat. Nos. 6,083,196, 6,050,988 and 6,091,975, and U.S. Patent Application Pub. No. 2002/0016562, which are incorporated by reference herein in their entirety. As will be appreciated by one having ordinary skill in the art, where a microprojection array is employed, the dose of the therapeutic agent that is delivered can also be varied or manipulated by altering the microprojection array (or patch) size, density, etc.

### DETAILED DESCRIPTION OF THE INVENTION

As indicated above, the present invention comprises compositions of and methods for formulating and delivering peptide therapeutic agents having enhanced physical stability, and wherein fibril formation is minimized and/or controlled. The compositions of and methods for formulating and delivering peptide, polypeptide and protein therapeutic agent formulations further allow for the minimization and/or control of fibril formation to yield a consistent and predictable composition viscosity. The compositions of and methods for formulating and delivering peptide, polypeptide and protein therapeutic agent formulations of the present invention further facilitate their incorporation into a biocompatible coating which can be employed to coat a stratum-corneum piercing microprojection, or a plurality of stratum-corneum piercing microprojections of a delivery device, for delivery of the biocompatible coating through the skin of a subject, thus providing an effective means of delivering the peptide therapeutic agents.

According to one embodiment, the present invention comprises a peptide therapeutic agent formulation wherein fibril formation is controlled and formulation viscosity regulated by the presence of at least two counterions in the peptide therapeutic agent formulation. The counterion mixture for the therapeutic peptides or polypeptides includes all those species that form pharmaceutically acceptable salts thereof. Thus, counterions may comprise weak or strong organic or inorganic acids or bases, surfactants, polymers, or other moieties having a net charge. For peptides or polypeptides possessing a net negative charge, the counterion mixture preferably possesses a net positive charge at the solution pH. For peptides or polypeptides possessing a net positive charge, the counterion mixture preferably possesses a net negative at the solution pH.

Generally, in the noted embodiments of the present invention, the amount of counterion mixture should be sufficient to neutralize the net charge of the peptide therapeutic agent.

Where two counterions are employed, a mole ratio of the two counterions is preferably in the range of about 0.2:1-5:1, more preferably, in the range of about 0.5:1-2:1. Where three or more counterions are employed, the mole ratio of any individual counterion to the molar sum of the others is preferably in the range of about 0.1:1-2.5:1, more preferably, in the range of about 0.25:1-1:1. The mole ratio of the counterion mixture to peptide is preferably in the range of about 2:1-30:1, more preferably, in the range of about 4:1-15:1.

It will be apparent to one having ordinary skill in the art that adding small amounts of a third counterion to a working mixture of two counterions that inhibit fibril formation of a given peptide would obviously result in a mole ratio of the third counterion to the molar sum of the others outside the working ranges indicated above. It is thus obvious that such an artifice is also in the scope of this application.

### Therapeutic Agents

A great number and variety of peptide, polypeptide, and protein therapeutic agents are known in the art to have therapeutic benefits when delivered appropriately to a patient having a condition upon which such therapeutic agents can exert a beneficial effect. These therapeutic agents comprise several very broad classes, including hormones, proteins, antigens, immunoglulins, repressors/activators, enzymes, among others.

Suitable hormones that can be employed within the scope of the present invention include protein hormones, such as insulin. As will be appreciated by one having ordinary skill in the art, the noted hormones are typically employed for treatment of diverse conditions and diseases, including cancer, metabolic diseases, pituitary conditions and menopause.

Initially, only a few peptides and proteins were considered capable of forming fibrils, although also short fragments of these particular proteins form fibrils. More recently, it has been established that even globular all-helical proteins, such as myoglobin, which normally do not give rise to fibrils, can be converted to fibrils if incubated under partly denaturing conditions (Fandrich, M., Fletcher, M. A., and Dobson, C. M. (2001) Nature 410, 165-166). This suggests that most proteins have the potential to form fibrils. In addition, it is documented that peptides as short as 4 residues can form fibrils (J. Biol. Chem., Vol. 277, Issue 45, 43243-43246, Nov. 8, 2002).

Thus, in one embodiment of the present invention, the compositions of therapeutic peptides, polypeptides and proteins includes at least one of the following agents that may form fibrils under usual or particular conditions: ACTH, amylin, angiotensin, angiogenin, anti-inflammatory peptides, BNP, calcitonin, endorphins, endothelin, GLIP, Growth Hormone Releasing Factor (GRF), hirudin, insulin, insulinotropin, neuropeptide Y, PTH and VIP.

Further specific examples of therapeutic agents include, without limitation, growth hormone release hormone (GHRH), octreotide, pituitary hormones (e.g., hGH), ANF, growth factors such as growth factor releasing factor (GFRF), bMSH, somatostatin, platelet-derived growth factor releasing factor, human chorionic gonadotropin, erythropoietin, glucagon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANF, ANP, ANP clearance inhibitors, antidiuretic hormone agonists, calcitonin gene related peptide (CGRP), IGF-1, pentigetide, protein C, protein S, thymosin alpha-1, vasopressin antagonists analogs, alpha-MSH, VEGF, PYY, and polypeptides and polypeptide analogs and derivatives derived from the foregoing.

In one embodiment ofthe invention, the peptide therapeutic agent possesses a net positive charge and the counterion mixture preferably possesses a net negative charge at the solution pH. Examples of positively-charged peptide therapeutic agents include TH9507 in the pH range 0-11, hCT in the pH range 0-8, hPTH (1-34) in the pH range 0-8.5, desmopressin in the pH range 0-11, hVEGF (1-121) in the pH range 0-6, and hBNP (1-33) in the range 0-10.

In the above embodiment, examples of counterions suitable for formulation with net positively charged peptides include, but are not limited to, acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate, and sulfonate. Preferably, the counterion mixture is added to the therapeutic agent formulation in an amount sufficient to neutralize the net charge of the peptide agent. However, an excess of counterion mixture (either as the acid or the conjugate acid-base) can be added to the peptide.

In another embodiment of the present invention, the peptide therapeutic agent possesses a net negative charge, and counterion mixture preferably possesses a net positive charge at the solution pH. Examples of negatively-charged peptide therapeutic agents include insulin in the pH range 6-14, VEGF in the pH range 6-14, and insulinotropin in the pH range 6-14.

In the above embodiment, examples of counterions suitable for formulation with net negatively charged peptides or polypeptides include, but are not limited to, sodium, potassium, calcium, magnesium, ammonium, monoethanolamine, diethanolamine, triethanolamine, tromethamine, lysine, histidine, arginine, morpholine, methylglucamine, and glucosamine. The counterion or counterion mixture is preferably added to the therapeutic agent formulation in an amount sufficient to neutralize the net charge of the peptide agent. However, an excess of counterion or counterion mixture (either as the base or the conjugate acid-base) can be added to the peptide.

In a particularly preferred embodiment of the present invention, the therapeutic peptide comprises a hormone. A particularly preferred hormone is Growth Hormone Releasing Factor (GRF) and analogs thereof, especially TH 9507. TH 9507 is a synthetic, 44-amino acid Growth Hormone Releasing Factor analog, which has been used to treat sleep disorders, multiple anabolic conditions, including muscle wasting in Chronic Obstructive Pulmonary Disease (COPD), immune and cognitive conditions. TH 9507 exhibits greater potency than its naturally-occurring counterpart, since TH 9507 has been stabilized by including a hydrophobic moiety which increases the peptide's plasma half-life.

As is well known in the art, GRF triggers secretion of the Growth Hormone (GH) by the pituitary, by binding to the pituitary receptor. In normal subjects, GH is released by the pituitary in pulsatile fashion; TH 9507 can also achieve this pulsatile release of GF.

In the noted embodiment, it is particularly preferred to stabilize the GRF with a mixture of acetate and chloride counterions. The mole ratio of acetate to chloride is preferably in the range of about 0.2:1-5:1, more preferably, in the range of about 0.5:1-2:1. The mole ratio of the counterion mixture to peptide is preferably in the range of about 2:1-30:1, more preferably, in the range of about 4:1-15:1.

In a preferred embodiment, the peptide therapeutic active agent and counterion (or counterion mixture) is formulated as a solution or suspension in an appropriate solvent. Suitable solvents include water, DMSO, ethanol, isopropanol, DMF, acetonitrile, N-methyl-2-pyrollidone, and mixtures thereof. In addition, the therapeutic peptide can be in solution or suspension in a polymeric vehicle, such as EVA or PLGA. As is known in the art, additional stabilizing additives, such as sucrose and trehalose, may be present in the formulation.

Various other additives that aid in the delivery, stability or efficacy of the peptide therapeutic agents of the present invention can also be added to the formulations of the invention; provided, the additive does not interact or interfere with the fibril formation-inhibiting counterions. Thus, the compositions and formulations of the present invention can contain suitable adjuvants, excipients, solvents, salts, surfactants, buffering agents and other components. Examples of such additives can be found in U.S. patent application Ser. Nos. 10/880,702 and 10/970,890, the disclosures of which are incorporated by reference herein.

In another embodiment of the present invention, fibril formation in a peptide therapeutic agent formulation is controlled and viscosity of the formulation regulated by the addition of an agent, compound or substance, whereby self association and/or self-assembly of the peptide agent is inhibited or controlled. Generally, the desired control of fibril formation and regulation of formulation viscosity can be achieved when the peptide is forced into a secondary conformation that is thermodynamically unfavorable to self-assembly.

As is well known in the art, energy transformations in systems of peptides, polypeptides and proteins are governed by the free energy equation shown below, where H represents enthalpy and S entropy:
ΔG=ΔH−TΔS Equation I:

Polypeptide folding, and, hence, capacity for self assembly, can be evaluated by the free energy Equation I. Additionally, distribution of ionic species in the peptide solution can be calculated. Equations for equilibrium calculations, which have been available for many years, are based on the classic equilibrium laws. They can be used successfully to calculate the net charge of polyelectrolytes, such as polypeptides as well as the pI of a protein.

As is known in the art, net charge and pI calculations are powerful tools for characterizing and purifying polypeptides. Nevertheless, these calculations do not yield direct information about the species present in solution at a specific pH.

In U.S. patent application Ser. No. 10/880,702, there is provided a method for deriving equations, and a computational algorithm, for describing the species distribution for any polyelectrolyte, provided that their pKₐ values are known. The disclosure of this application is fully incorporated by reference herein.

In additional embodiments of the present invention, the peptide therapeutic agents, which have been stabilized by minimizing or eliminating fibril formation, are formulated as a solution or suspension, and then can be dried, freeze-dried (or lyophilized), spray dried or spray-freeze dried to stabilize for storage.

In another preferred embodiment of the present invention, the peptide therapeutic agent formulations, which have been stabilized by minimizing or eliminating fibril formation, are included in biocompatible coating formulations used to coat a stratum-cornuem piercing microprojection, or plurality of a stratum-corneum piercing microprojections, or an array thereof, or delivery device, for delivery of the peptide therapeutic agent through the skin of a patient. Compositions of and methods for formulating biocompatible coatings are described in U.S. Patent Application Pub. No. 2002/0177839 to Cormier et al; U.S. Patent Application Pub. No. 2004/0062813 to Cormier et al and U.S. Patent Application Pub. No. 2002/0132054 to Trautman et al, the disclosures of which are incorporated herein by reference.

For peptide therapeutic agent formulations, particularly those therapeutic agents which comprise or include relatively high molecular weight polypeptides or proteins, it is preferred to formulate the biocompatible coating containing the therapeutic agent, such that a water-soluble, biocompatible polymer, is attached to, or associated with, the polypeptide or protein. A particularly preferred method is to form a conjugate of the polymer with the polypeptide or protein. The attachment of a polymer, such as PEG, to proteins and polypeptides typically results in improved solubility, improved physical and chemical stability, lower aggregation tendency and enhanced flow characteristics. Compositions of and methods for formulating biocompatible coatings having polymer conjugates of protein and polypeptide therapeutic agents are disclosed in U.S. patent application Ser. No. 10/972,231, the disclosures of which is incorporated herein by reference.

Other compositions of and methods for formulating and delivering protein-based therapeutic agent formulations are disclosed in U.S. Patent Application No. 60/585,276, filed Jul. 1, 2004, the disclosure of which is incorporated by reference herein. The noted application discloses compositions of and methods for formulating hormone therapeutic agents having a desired pharmacokinetic delivery profile, as well as the formulation of biocompatible coatings therewith.

In accordance with one embodiment of the invention, a method for delivering stable peptide therapeutic agent formulations comprises the following steps: (i) providing a microprojection member having a plurality of microprojections, (ii) providing a stabilized formulation of peptide therapeutic agent; (iii) forming a biocompatible coating formulation that includes the formulation of stabilized peptide therapeutic agent, (iv) coating the microprojection member with the biocompatible coating formulation to form a biocompatible coating; (v) stabilizing the biocompatible coating by drying; and (vi) applying the coated microprojection member to the skin of a subject.

FIG. 1 illustrates one embodiment of a stratum cornuem-piercing microprojection array for use with the compositions and methods for formulating and delivering of the present invention. As shown in FIG. 1, the microprojection array 5 includes a plurality of microprojections 10. The microprojections 10 extend at substantially a 90 degree angle from a sheet 12 having openings 14. As shown in FIG. 5, the sheet 12 can be incorporated in a delivery patch including a backing 15 for the sheet 12. The backing 15 can further include an adhesive 16 for adhering the backing 15 and microprojection array 5 to a patient's skin. In this embodiment, the microprojections 10 are formed by either etching or punching a plurality of microprojections 10 out of a plane of the sheet 12.

The microprojection array 5 can be manufactured of metals, such as stainless steel, titanium, nickel titanium alloys, or similar biocompatible materials, such as plastics. In a preferred embodiment, the microprojection array is constructed of titanium. Metal microprojection members are disclosed in Trautman et al., U.S. Pat. No. 6,038,196; Zuck U.S. Pat. No. 6,050,988; and Daddona et al., U.S. Pat. No. 6,091,975, the disclosures of which are herein incorporated by reference.

Other microprojection members that can be used with the present invention are formed by etching silicon, by utilizing chip etching techniques or by molding plastic using etched micro-molds. Silicon and plastic microprojection members are disclosed in Godshall et al., U.S. Pat. No. 5,879,326, the disclosure of which is incorporated herein by reference.

With such microprojection devices, it is important that the biocompatible coating having the peptide therapeutic agent is applied to the microprojections homogeneously and evenly, preferably limited to the microprojections themselves. This enables dissolution of the peptide therapeutic agent in the interstitial fluid once the device has been applied to the skin and the stratum cornuem pierced. Additionally, a homogeneous coating provides for greater mechanical stability both during storage and during insertion into the skin. Weak and/or discontinuous coatings are more likely to flake off during manufacture and storage, and to be wiped of the skin during application.

Additionally, optimal stability and shelf life of the agent is attained by a biocompatible coating that is solid and substantially dry. However, the kinetics of the coating dissolution and agent release can vary appreciably depending upon a number of factors. It will be readily appreciated that in addition to being storage stable, the biocompatible coating should permit desired release of the therapeutic agent.

Depending on the release kinetics profile, it may be necessary to maintain the coated microprojections in piercing relation with the skin for extended periods of time (e.g., up to about 8 hours). This can be accomplished by anchoring the microprojection member to the skin using adhesives or by using anchored microprojections, such as described in U.S. Pat. No. 6,230,051, to Cormier et al, the disclosure of which is incorporated by reference herein in its entirety.

The compositions of and methods for formulating of the present invention provide the additional benefit of permitting the formulation viscosity to be controlled, which facilitates applying the therapeutic agent (or a biocompatible coating containing the therapeutic agent) onto a microprojection delivery device such as those having at least one stratum-cornuem piercing microprojection, and preferably a plurality of such stratum-cornuem piercing microprojections. With such devices, the viscosity of the coating formulation should be controlled to enable the release kinetics necessary to ensure adequate flux of the therapeutic agent. At the same time, some formulation viscosity can aid in manufacturing such microprojection devices, since some formulation viscosity allows more coating to be deposited upon the available microprojection surface area of the microprojection member.

Compositions of and methods for formulating biocompatible coatings are described, for example, in U.S. Patent Application Pub. Nos. 2002/0128599, 2002/0177839 and 2004/0115167, the disclosures of which are incorporated herein by reference.

In one embodiment of the present invention, a dip-coating process is employed to coat the microprojections by partially or totally immersing the microprojections into the biocompatible coating solution containing the stable peptide therapeutic agent formulation. Alternatively, the entire device can be immersed into the biocompatible coating solution.

In many instances, the stable therapeutic agent within the coating can be very expensive. Therefore, it may be preferable to only coat the tips of the microprojections. Microprojection tip coating apparatus and methods are disclosed in Trautman et al., U.S. Patent Application Pub. No. 2002/0132054. The noted publication discloses a roller coating mechanism that limits the coating to the tips of the microprojection.

As described in the Trautman et al publication, the coating device only applies the coating to the microprojections and not upon the substrate/sheet that the microprojections extend from. This may be desirable in the case where the cost of the active (or beneficial) agent is relatively high and therefore the coating containing the beneficial agent should only be disposed onto parts of the microprojection array that will pierce beneath the patient's stratum corneum layer.

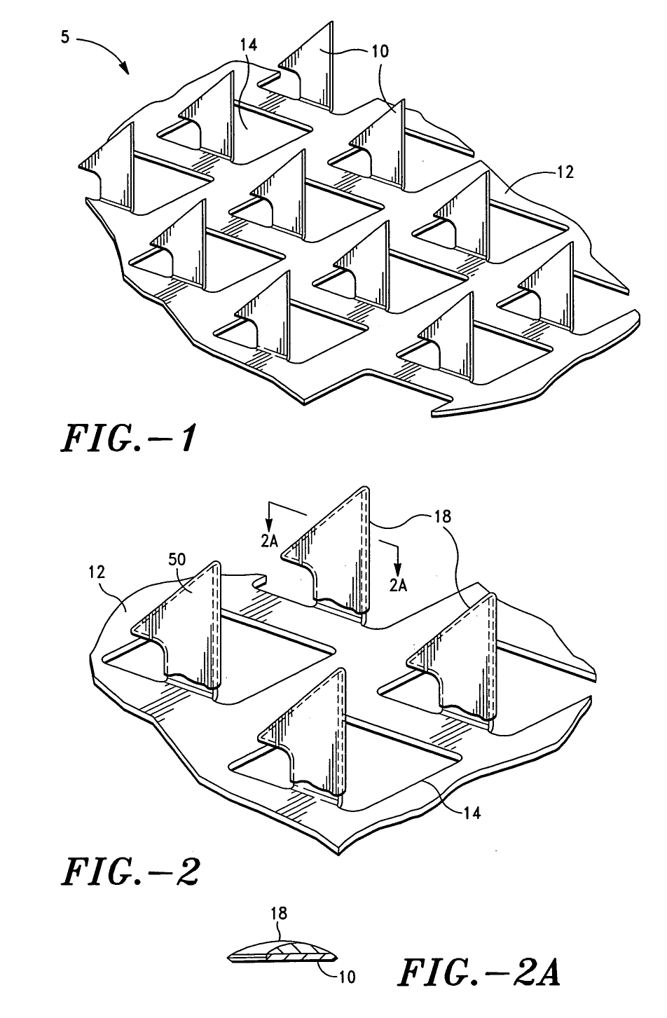

The noted coating technique has the added advantage of naturally forming a smooth coating that is not easily dislodged from the microprojections during skin piercing. The smooth cross section of the microprojection tip coating is more clearly shown in FIG. 2A.

Other coating techniques, such as microfluidic spray or printing techniques, can also be used to precisely deposit a coating 18 on the tips of the microprojections 10, as shown in FIG. 2.

Other coating methods that can be employed in the practice of the present invention include spraying the coating solution onto the microprojections. Spraying can encompass formation of an aerosol suspension of the coating composition. In one embodiment, an aerosol suspension forming a droplet size of about 10 to about 200 picoliters is sprayed onto the microprojections and then dried.

The microprojections 10 can further include means adapted to receive and/or increase the volume of the coating 18 such as apertures (not shown), grooves (not shown), surface irregularities (not shown), or similar modifications, wherein the means provides increased surface area upon which a greater amount of coating may be deposited.

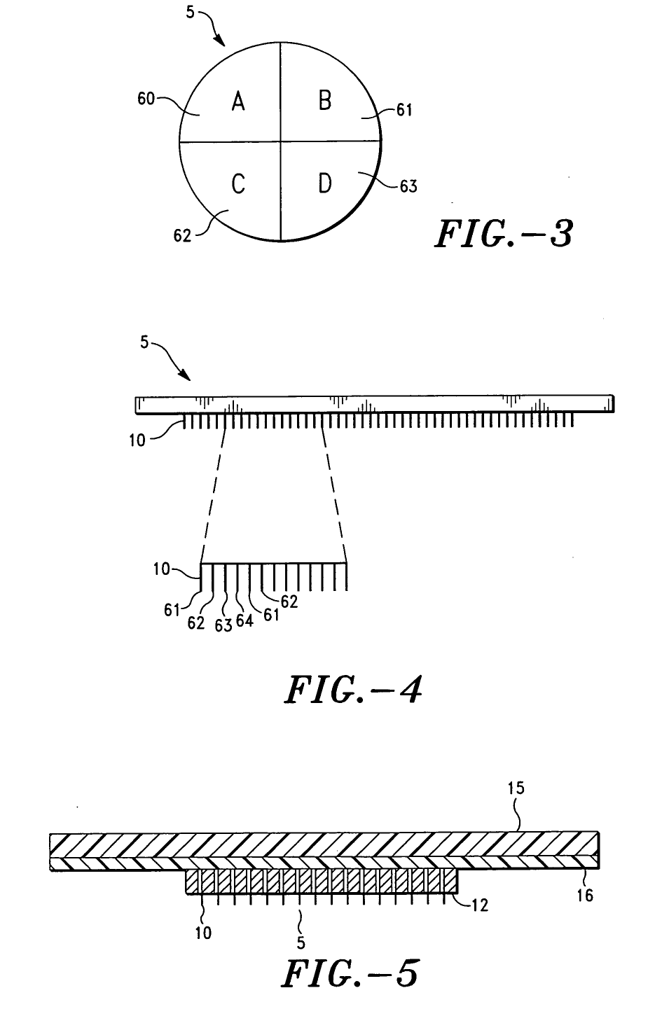

Referring now to FIGS. 3 and 4, there is shown an alternative embodiment of a microprojection array 5. As shown in FIG. 3, the microprojection array 5 may be divided into portions illustrated at 60-63, wherein a different coating is applied to each portion, thereby allowing a single microprojection array to be utilized to deliver more than one beneficial agent during use.

Referring now to FIG. 4, there is shown a cross-sectional view of the microprojection array 5, wherein a “pattern coating” has been applied to the microprojection array 5. As shown, each of the microprojections 10 can be coated with a different biocompatible coating and/or a different therapeutic agent, as indicated by reference numerals 61-64. That is, separate coatings are applied to the individual microprojections 10. The pattern coating can be applied using a dispensing system for positioning the deposited liquid onto the surface of the microprojection array. The quantity of the deposited liquid is preferably in the range of 0.1 to 20 nanoliters/microprojection. Examples of suitable precision-metered liquid dispensers are disclosed in U.S. Pat. Nos. 5,916,524, 5,743,960, 5,741,554 and 5,738,728, the disclosures of which are incorporated herein by reference.

Microprojection coating solutions can also be applied using ink jet technology using known solenoid valve dispensers, optional fluid motive means and positioning means which are generally controlled by use of an electric field. Other liquid dispensing technology from the printing industry or similar liquid dispensing technology known in the art can be used for applying the pattern coating of this invention.

In yet another preferred embodiment, the process of applying a biocompatible coating containing a peptide therapeutic agent of the invention to at least one stratum-cornuem piercing microprojection of a microprojection member, more preferably, to a plurality of such stratum-corneum piercing microprojections, includes the step of further stabilizing the biocompatible coating by drying. The drying step can occur at ambient (room) temperatures and conditions, or can employ temperatures in the range of 4 to 50° C.

Suitable drying methods and apparatus are disclosed in U.S. Patent Application No. 60/572,861, filed May 19, 2004, the disclosure of which is incorporated herein by reference.

According to the invention, a multitude of peptide therapeutic active agents can be subjected to the formulation process and methods of the invention to provide highly stable peptide formulations. In a preferred embodiment of the invention, the therapeutic agent comprises a hormone, especially GRF or an analog thereof, such as TH 9507.

As discussed in detail below, the present invention firther provides for methods for evaluating, predicting and inhibiting peptide self assembly, based upon charge distribution, stoichometric and thermodynamic considerations. Indeed, self-association is a crystallization-like process. Further, crystals only occur with compounds that can self-associate in repetitive patterns, which is only possible if the basic units, or molecules, are identical to each other. Thus, the introduction of several counterions in a peptide formulation would make the formulation behave like a mixture of different peptides, which would render self-assembly very difficult. For example, each molecule of TH 9507 at a pH 5.5 has four positive charges and can therefore associate with four negatively charged counterion molecules. If acetate (or chloride) is the only counterion, only one peptide salt can form, and self association is favored. If acetate and chloride are present in equimolar amounts, then 16 different peptide salts are present in solution, and self association is prevented.

### EXAMPLES

The following studies and examples illustrate the formulations, methods and processes of the invention. The examples are for illustrative purposes only and are not meant to limit the scope of the invention in any way.

### Example 1

### Prior Art

A first lot of the GRF analog TH 9507 was prepared by Bachem AG. This lot included an acetate counterion at a molar ratio of about 6.5 to the peptide.

The peptide conformation in aqueous solution was found by FTIR to be mostly an alpha helix. The solution physical properties were also found to be unstable.

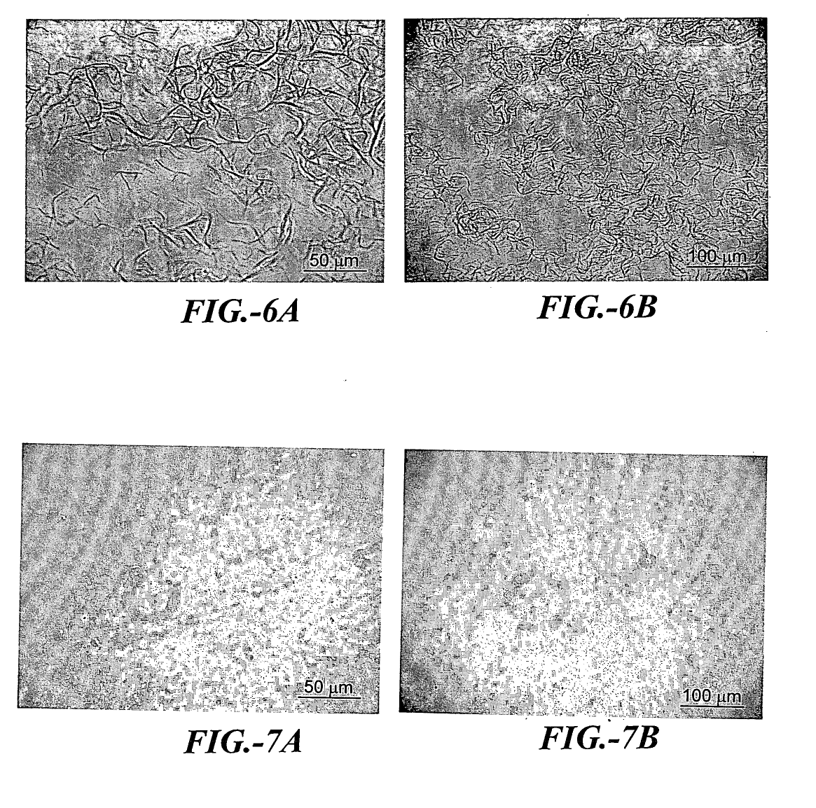

Solution viscosity increased as a function of storage time and fibrils started to appear in solution after only a few hours at room temperature (about 20° C.). FIGS. 6A and 6B are photomicrographs taken 6 hours after sample preparation. The noted photomicrographs visually demonstrate the formation of fibrils.

In this solution, fibril formation was found to be dependant upon the peptide concentration. At peptide concentrations of 1% and below, no fibril formation was observed. At peptide concentrations of 2% through 25%, observable fibril formation resulted within a few hours.

### Example 2

A second lot of the Growth Hormone Releasing Factor (GRF) analog TH 9507 was prepared by Bachem AG. This lot was found to contain equimolar amounts of the counterions acetate and chloride. The counterion mixture was present in a mole ratio to the TH 9507 in the range of about 4 to 1.

The peptide conformation in the solution was found by FTIR to present some beta sheet characteristics. Solutions of up to 7.5% peptide were found to be very stable (i.e., no fibril formation was observed during storage of the solution at room temperature).

Solution viscosity did not change after storage for several days at room temperature (about 20° C.) or after storage at 4° C. Neither storage condition resulted in visible fibrils in the formulation. FIGS. 7A and 7B are photomicrographs of samples of this formulation.

### Example 3

From the second lot of TH 9507 (Example 2), the hydrochloride form was synthesized by extensive dialysis of 10 mg solutions of TH 9507 acetate against a 10^{—4} M solution of hydrochloric acid. The resultant salt solution was subsequently lyophilized, yielding TH 9507 hexahydrochloride. This salt form was found to behave similarly to the first lot of TH9507 (i.e., the acetate salt of TH 9507). Thus, as in the acetate salt sample (Example 1), viscosity increased as a function of storage time and fibrils started to appear in solution after only a few hours at room temperature (about 20° C.).

### Example 4

To an aqueous solution of the TH 9507 acetate salt (Example 1), chloride ions (as sodium chloride) were added in increasing amounts. The addition of such chloride ions resulted in a decrease or increase rate of fibril formation, depending on the ratio of concentration of chloride ion to the TH 9507 acetate salt. The results are shown in Table I.

It can be seen that for solutions of the acetate salt of TH 9507, as added chloride ion nears an equimolar concentration to that of acetate, the solution viscosity is relatively low and stable, and fibril formation is minimal. Where there is a molar excess of acetate or chloride, the solution viscosity increases, and changes over time. Fibril formation is evident in such formulations.

TABLE 1

| | | | | | | Mole ratio | | | | | |
|---|---|---|---|---|---|---|---|---|---|---|---|
| | Concentration (M) | | | Mole ratio | | counterion/GRF | | | Solution properties | | |
| Ref | GRF | B | Cl | D/B | B/D | D | Cl | D + B | 0 h | 6 h | 72 h |
| 1 | 0.016 | 0.106 | 0 | | 0.0 | 6.5 | 0 | 6.6 | C/LV | F/LV | A/HV |
| 2 | 0.016 | 0.106 | 0.016 | 6.6 | 0.2 | 6.5 | 1 | 7.6 | C/LV | F/LV | A/HV |
| 3 | 0.016 | 0.106 | 0.033 | 3.2 | 0.3 | 6.5 | 2 | 8.7 | C/LV | F/LV | A/IV |
| 4 | 0.016 | 0.106 | 0.049 | 2.2 | 0.5 | 6.5 | 3 | 9.7 | C/LV | C/LV | C/IV |
| 5 | 0.016 | 0.106 | 0.073 | 1.5 | 0.7 | 6.5 | 4.5 | 11.2 | C/LV | C/LV | C/IV |
| 6 | 0.016 | 0.106 | 0.098 | 1.1 | 0.9 | 6.5 | 6 | 12.8 | C/LV | C/LV | A/IV |
| 7 | 0.016 | 0.106 | 0.13 | 0.8 | 1.2 | 6.5 | 8 | 14.8 | C/LV | C/LV | A/HV |
| 8 | 0.016 | 0.034 | 0.031 | 1.1 | 0.9 | 2.1 | 2 | 4.1 | C/IV | C/IV | C/IV |
| 9 | 0.016 | 0 | 0.096 | 0.0 | | 0 | 6 | 6.0 | C/LV | F/LV | C/HV |

C: Clear solution
F: Fibrils
A: Large aggregates
B: Chloride
LV: Low viscosity
IV: Intermediate viscosity
HV: High viscosity
D: Acetate

### Example 5

Hydrochloride and mesylate salts of TH 9507 were prepared by extensive dialysis of 10 mg solutions of TH 9507 acetate against 10^{—4} M solutions of hydrochloric acid and methanesulfonic acid, respectively. The resultant salt solutions were subsequently lyophilized, yielding TH 9507 hexahydrochloride, and TH 9507 hexamesylate. From these, 50 mg/mL peptide salt solutions were prepared, containing the following ratios of TH 9507 hexahydrochloride to TH 9507 hexamesylate: 1, 0.8, 0.67, 0.57, 0.5, 0.43, 0.33, 0.2, 0.

Visual and microscopic visualization following storage of the solutions at 4° C. revealed that fibril formation occurred readily with the hexahydrochloride salt. Fibril formation was inhibited by the presence of the hexamesylate salt at a ratio to the TH 9507 as low as 0.2.

The data set forth above reflects that peptide conformation and self-assembly into fibrils can be controlled by the addition to the peptide solution of an appropriate counterion, or counterion mixture. The presence of two or more counterions (for example chloride and acetate) results in inhibition of fibril formation.

In accordance with the present invention, the self-association exhibited by certain polypeptides can be thought of as a crystallization-like process. As will be appreciated by one having ordinary skill in the art, crystals only occur with compounds that can self-associate in repetitive patterns, which is only possible if the basic units, or molecules, are identical to each other. The addition of appropriate counterions to a peptide formulation makes the formulation behave like a mixture of different peptides, which renders peptide self-assembly very difficult.

The present invention thus has utility in improving the physical stability, especially the viscosity stability of peptide therapeutic agent formulations. Fibril formation may occur within a few hours and jeopardize manufacturability of the final product, in particular, those products for which the viscosity of the formulation is important. Thus, formulation viscosity control is important for therapeutic agent formulations included in a biocompatible coating coated onto a plurality of stratum-corneum piercing microprojections of a microprojection member or device. In addition, regardless of whether the peptide therapeutic agent formulation is liquid, solid, semi-solid or dry, mitigation or elimination of fibril formation by the compositions of and methods for formulating and delivering of the present invention result in a maximal or optimal shelf life for the product.

While the forgoing embodiments describe delivery of the stable peptide therapeutic agent formulations via a biocompatible coating applied to stratum-corneum piercing microprojection, or a plurality of stratum-corneum piercing microprojections of a delivery device, the compositions of and method for formulating and delivering the peptide therapeutic agent formulations of the present invention can be employed with various other deliver schemes, systems, devices and protocols, capable of delivering the therapeutic agents in liquid, solid, or semi-solid, and dry form. Thus, the compositions and formulations of the present invention can be employed with oral delivery (bolus or pattern), infusion, injection, implant, aerosol, passive and active transdermal, and other delivery modes, systems, devices and formulations.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A composition for coating a transdermal delivery device having stratum corneum-piercing microprojections comprising a formulation of a therapeutically effective amount of a peptide agent and at least one counterion to substantially reduce fibril formation and viscosity variation in the composition.

2. A composition of claim 1, wherein the peptide agent is in a secondary conformation that is thermodynamically unfavorable to self-association.

3. A composition of claim 1, wherein the peptide agent is associated with a water-soluble, biocompatible polymer.

4. A composition of claim 1, wherein said peptide agent is selected from the group consisting of growth hormone release hormone (GHRH), growth hormone release factor (GHRF), insulin, insulinotropin, calcitonin, octreotide, endorphin, growth factors such as growth factor releasing factor (GFRF), bMSH, platelet-derived growth factor releasing factor, pituitary hormones (hGH), ANF, ACTH, amylin, angiotensin, angiogenin, anti-inflammatory peptides, BNP, endothelin, GLIP, hirudin, neuropeptide Y, PTH, VIP, somatostatin, human chorionic gonadotropin, erythropoietin, gluacgon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANP, ANP clearance inhibitors, antidiuretic hormone agonists, calcitonin gene related peptide (CGRP), IGF-1, pentigetide, protein C, protein S, thymosin alpha-1, alpha-MSH, VEGF, PYY, and peptide analogs and derivatives derived from a peptide agent in the group.

5. A composition of claim 1, wherein said peptide agent is Growth Hormone Release Factor or an analog or derivative of Growth Hormone Release Factor.

6. A composition of claim 1, wherein the at least one counterion is present in a sufficient amount to neutralize the net charge of the peptide agent at the formulation pH.

7. A composition of claim 1, wherein the peptide agent has a net positive charge and the at least one counterion has a net negative charge at the formulation pH.

8. A composition of claim 1, wherein the peptide agent has a net negative charge and the at least one counterion has a net positive charge at the formulation pH.

9. A composition of claim 1, wherein the at least one counterion is a weak or strong, inorganic or inorganic, acid or base, surfactant, polymer, or other moiety having a net charge.

10. A composition of claim 7, wherein the at least one counterion is selected from the group consisting of acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate, and sulfonate.

11. A composition of claim 8, wherein the at least one counterion is selected from the group consisting of sodium, potassium, calcium, magnesium, ammonium, monoethanolamine, diethanolamine, triethanolamine, tromethamine, lysine, histidine, arginine, morpholine, methylglucamine, and glucosamine.

12. A composition of claim 1, wherein the mole ratio of the at least one counterion to the peptide agent is in the range of about 2:1to 30:1.

13. A composition of claim 1, wherein the peptide agent is Growth Hormone Release Factor or an analog or derivative of Growth Hormone Release Factor and the counterion is acetate or chloride.

14. A composition of claim 1, wherein there is a mixture of counterions.

15. A composition of claim 14, wherein the mixture of counterions includes two counterions and the mole ratio of the two counterions is in the range of about 0.2:1 to 5:1.

16. A composition of claim 14, wherein the mixture of counterions includes three or more counterions and the mole ratio of any individual counterion to the molar sum of the other counterions is in the range of about 0.1:1 to 2.5:1.

17. A composition of claim 1, wherein the peptide agent is Growth Hormone Release Factor or an analog or derivative of Growth Hormone Release Factor and the counterions include acetate and chloride.

18. A composition of claim 1, further comprising a transdermal delivery device having at least one microprojection configured to pierce the stratum cornuem.

19. A composition of claim 18, wherein said composition is coated on said microprojection and dried.

20. A method for applying a biocompatible coating to a transdermal delivery device that has at least one stratum cornuem-piercing microprojection comprising the steps of:
providing a formulation of a peptide agent and at least one counterion to substantially reduce fibril formation and viscosity variation in the composition;
applying said formulation to the device; and
drying said formulation.

21. A method of claim 20, wherein the formulation is applied to at least one microprojection.

22. A method of claim 20, further comprising the step of subjecting the formulation to drying, freeze-drying, spray-drying or spray freeze-drying prior to application to the device.

23. A method of claim 20, further comprising the step of forming a biocompatible coating formulation that includes the formulation of a peptide and at least one counterion.

24. A method of claim 20, wherein the peptide agent is in a secondary conformation that is thermodynamically unfavorable to self-association.

25. A method of claim 20, wherein the peptide agent is associated with a water-soluble, biocompatible polymer.

26. A method of claim 20, wherein said peptide agent is selected from the group consisting of growth hormone release hormone (GHRH), growth hormone release factor (GHRF), insulin, insulinotropin, calcitonin, octreotide, endorphin, growth factors such as growth factor releasing factor (GFRF), bMSH, platelet-derived growth factor releasing factor, pituitary hormones (hGH), ANF, ACTH, amylin, angiotensin, angiogenin, anti-inflammatory peptides, BNP, endothelin, GLIP, hirudin, neuropeptide Y, PTH, VIP, somatostatin, human chorionic gonadotropin, erythropoietin, gluacgon, hirulog, interferon alpha, interferon beta, interferon gamma, interleukins, granulocyte macrophage colony stimulating factor (GM-CSF), granulocyte colony stimulating factor (G-CSF), menotropins (urofollitropin (FSH) and LH)), streptokinase, tissue plasminogen activator, urokinase, ANP, ANP clearance inhibitors, antidiuretic hormone agonists, calcitonin gene related peptide (CGRP), IGF-1, pentigetide, protein C, protein S, thymosin alpha-1, alpha-MSH, VEGF, PYY, and peptide analogs and derivatives derived from a peptide agent in the group.

27. A method of claim 20, wherein said peptide agent is Growth Hormone Release Factor or an analog or derivative of Growth Hormone Release Factor.

28. A method of claim 20, wherein the at least one counterion is present in a sufficient amount to neutralize the net charge of the peptide agent at the formulation pH.

29. A method of claim 20, wherein the peptide agent has a net positive charge and the at least one counterion has a net negative charge at the formulation pH.

30. A method of claim 20, wherein the peptide agent has a net negative charge and the at least one counterion has a net positive charge at the formulation pH.

31. A method of claim 20, wherein the at least one counterion is a weak or strong, inorganic or inorganic, acid or base, surfactant, polymer, or other moiety having a net charge.

32. A method of claim 29, wherein the at least one counterion is selected from the group consisting of acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate, and sulfonate.

33. A method of claim 30, wherein the at least one counterion is selected from the group consisting of sodium, potassium, calcium, magnesium, ammonium, monoethanolamine, diethanolamine, triethanolamine, tromethamine, lysine, histidine, arginine, morpholine, methylglucamine, and glucosamine.

34. A method of claim 20, wherein the mole ratio of the at least one counterion to the peptide agent is in the range of about 2:1 to 30:1.

35. A method of claim 20, wherein the peptide agent is Growth Hormone Release Factor or an analog or derivative of Growth Hormone Release Factor and the counterion is acetate or chloride.

36. A method of claim 20, wherein there is a mixture of counterions.

37. A method for transdermally delivering a peptide agent comprising the steps of:
providing a transdermal delivery device having at least one stratum cornuem-piercing microprojection, the microprojection including a biocompatible coating comprising a dried formulation of said peptide agent and at least one counterion to substantially reduce fibril formation and viscosity variation in the coating; and
applying said delivery device to a patient to deliver said biologically active agent.
